(19) United States
(12) Patent Application Publication
(10) Pub. No.: US 2015/0342506 A1
(43) Pub. Date: Dec. 3, 2015

(54) SYSTEM AND METHOD FOR EVALUATING HYPERMONITORING OF TINNITUS

(71) Applicant: SOUNDCURE, INC, San Jose, CA (US)

(72) Inventor: Jeffrey Carroll, Corona, CA (US)

(73) Assignee: SOUNDCURE, INC., San Jose, CA (US)

(21) Appl. No.: 14/759,961

(22) PCT Filed: Jan. 11, 2013
(86) PCT No.: PCT/US13/21260

§ 371 Date: Jul. 9, 2015

(51) Int. Cl. A61B 5/12; A61B 5/00

(52) U.S. Cl. CPC A61B 5/128; A61B 5/72; A61B 5/742; A61B 5/7475; A61B 2560/0475

(57) ABSTRACT

To evaluate hypermonitoring of a patient, a processor controls an audiometer or computer software interface and executes instructions in a memory device which present various tones to the patient to determine a pitch match tone, a clinical loudness match, a subjective loudness and a subjective perceived loudness relative to the patient's tinnitus. A display and input device provide an interface between the processor and an operator.

### BACKGROUND

The present invention generally relates to systems and methods for assessing a person's tinnitus.

A standard manual tinnitus evaluation, as described by CPT (current procedural terminology) code 92625, is a manual process including a tinnitus pitch match, tinnitus loudness match and masking studies, most commonly including both a minimum masking level and an evaluation of residual inhibition. The pitch match is the closest pure tone to the patient's perceived tinnitus in each ear. The loudness match is the loudness of the tinnitus, at the pitch match, in units of dB at the hearing level (HL). The minimum masking level is the loudness of white noise or narrow band noise that completely covers the patient's tinnitus. The residual inhibition is the phenomenon where tinnitus disappears following masking and occurs in some patients. It is evaluated by completely covering their tinnitus with the masker and then turning it off. Whether this occurs and for how long it occurs is determined as part of the standard evaluation.

### SUMMARY

There are limitations regarding the manual evaluation and particularly the loudness match. The vast majority of tinnitus patients match their tinnitus to 10 dB or less above their threshold. A threshold is the softest sound a patient can hear. For reference, normal hearing is defined as a threshold softer than 20 dB HL for any given tone (in the range 250-8000 Hz). Normal upper loudness levels (ULL), or the level at which you would not want to go above, is between 100 and 120 dB HL. Some benchmarks include 30 dB (whisper soft), 55-65 dB (conversational speech), 70-80 dB (freeway traffic), and 110 dB (power tools). The systems and methods of the invention address this and other limitations of the manual tinnitus evaluation.

An aspect of the invention is directed to a system for use with an audiometer for evaluating hypermonitoring of tinnitus of a patient. The system comprises a memory device for storing executable instructions, a processor, a display and an input device for use by the operator for providing input information to the processor. The processor is adapted for controlling the audiometer, adapted for accessing the memory device and adapted to execute the executable instructions stored on the memory device. The display is driven by the processor for providing screen shots to an operator of the system. The executable instructions stored in the memory device include:
instructions for determining a pitch match tone (PMT) of the patient's tinnitus;
instructions for determining a clinical loudness match (CLM) of the patient's tinnitus;
instructions 20 for determining a subjective loudness (SL) of the patient's tinnitus; and
instructions for determining a subjective perceived loudness (SPL) of the patient's PMT at the CLM.

Another aspect of the invention is directed to a processor executable method for use with an audiometer for evaluating hypermonitoring of tinnitus of a patient. The method includes processor executable instructions for controlling the audiometer. The processor executable method includes:

determining a pitch match tone (PMT) of the patient's tinnitus;

determining a clinical loudness match (CLM) of the patient's tinnitus;

determining a subjective loudness (SL) of the patient's tinnitus; and

determining a subjective perceived loudness (SPL) of the patient of the PMT at the CLM.

Yet another aspect of the invention is directed to tangible computer readable storage media storing processor executable instructions for use with an audiometer for evaluating hypermonitoring of tinnitus of a patient. The processor executable instructions are adapted for controlling the audiometer. The media comprises:
instructions for determining a pitch match tone (PMT) of the patient's tinnitus;
instructions for determining a clinical loudness match (CLM) of the patient's tinnitus;
instructions for determining a subjective loudness (SL) of the patient's tinnitus; and
instructions for determining a subjective perceived loudness (SPL) 208 of the patient of the PMT at the CLM.

Other objects and features will be in part apparent and in part pointed out hereinafter.

### BRIEF DESCRIPTION OF THE DRAWINGS

FIG. 1 is a block diagram of a system and method according to the invention.

FIG. 2 is a block diagram of another system and method according to the invention.

FIG. 3 is a flow chart of a system and method of the invention.

FIG. 4 is a screen shot of a system and method of the invention relating to pitch match thresholds.

FIG. 5 is a screen shot of a system and method of the invention relating to a subjective loudness determination.

Corresponding reference characters indicate corresponding parts throughout the drawings.

### DETAILED DESCRIPTION

Systems, methods and tangible computer readable mediums are described in which data is collected for a patient who has tinnitus so that the perceived loudness of the patient's tinnitus can be compared to a more objective loudness determination of the patient's tinnitus. A discrepancy between the perceived loudness and more objective loudness is defined as hypermonitoring. This system and method implements a tinnitus evaluation that is performed automatically by an audiologist using a computer/processor controlling an audiometer. The value of this test is both in counseling a patient about how sound therapy works, in that it corrects this perceptual mismatch when present, and can be used to counsel patients about sound therapy. For example, patients who are hypermonitoring can achieve improvement through the process of habituation as this perceptual mismatch is corrected.

The methods and systems can be embodied in software to allow for an easy automated way of implementation. The methods and systems provide a general tool in any implementation.

A patient with normal hearing (threshold=20 dB) and tinnitus at 10 dB above threshold (tinnitus loudness match=30 dB) would have tinnitus that is equivalent to whisper soft. Patients with hearing loss often have a reduced range between threshold and normal upper loudness levels (ULL) so the loudness of a fixed number above threshold is no longer known. For example, if a patient has a threshold of 50 dB and a ULL of 100 dB, a 60 dB hearing level (HL) may not be whisper soft to the patient; it could be perceived as loud. Further, often with hearing loss the ULL is decreased so that a patient may have a threshold of 50 dB and a ULL of 85 dB, compounding the above problem.

Some patients experience sound sensitivity either across the entire frequency range or at a specific frequency. This sensitivity also compresses the usable hearing range making the dB above threshold an unknown (e.g., a patient has a threshold of 20 dB but a ULL of 40 dB). In this case, 30 dB would also not likely be whisper soft.

The methods and systems described herein overcome these limitations by evaluating the extent of hypermonitoring, and/or by comparing the perceived loudness of the tinnitus loudness match to the patient's perceived loudness of his own tinnitus. Thus, a comparison of the subjective rated loudness of the patient's tinnitus loudness match to the patient's subjective rating of his own tinnitus is provided.

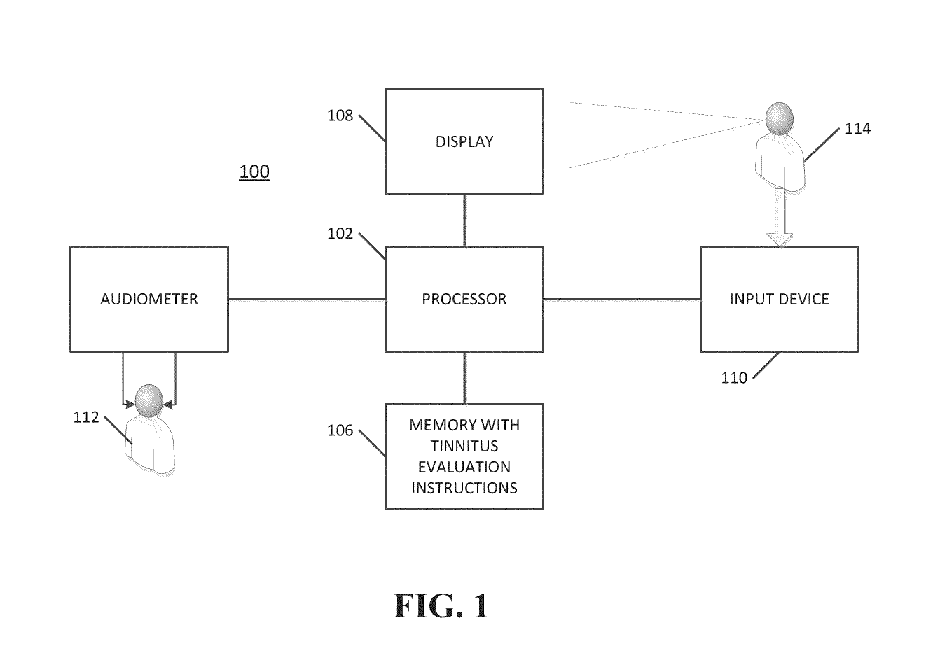

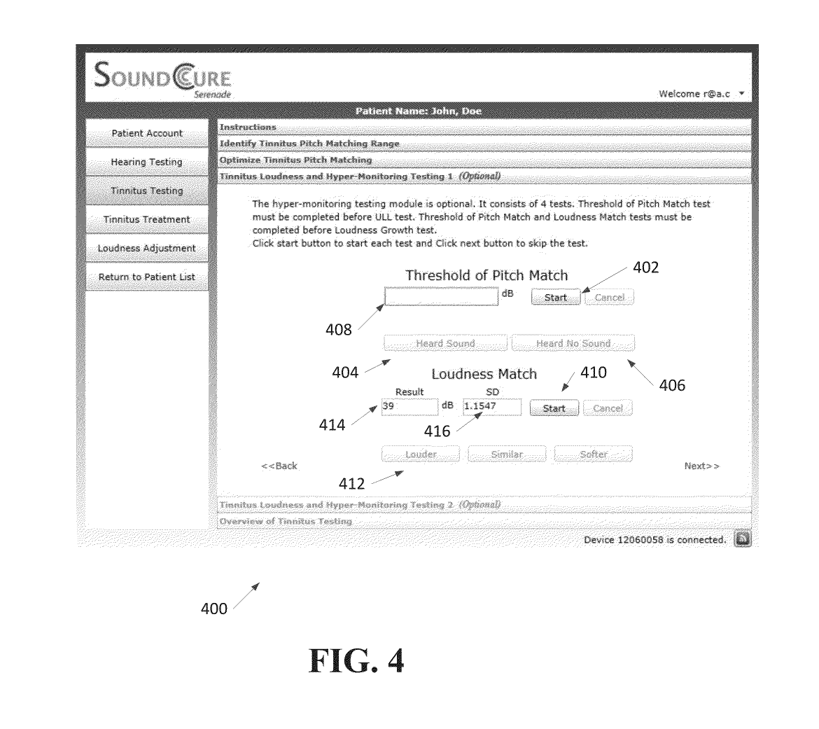

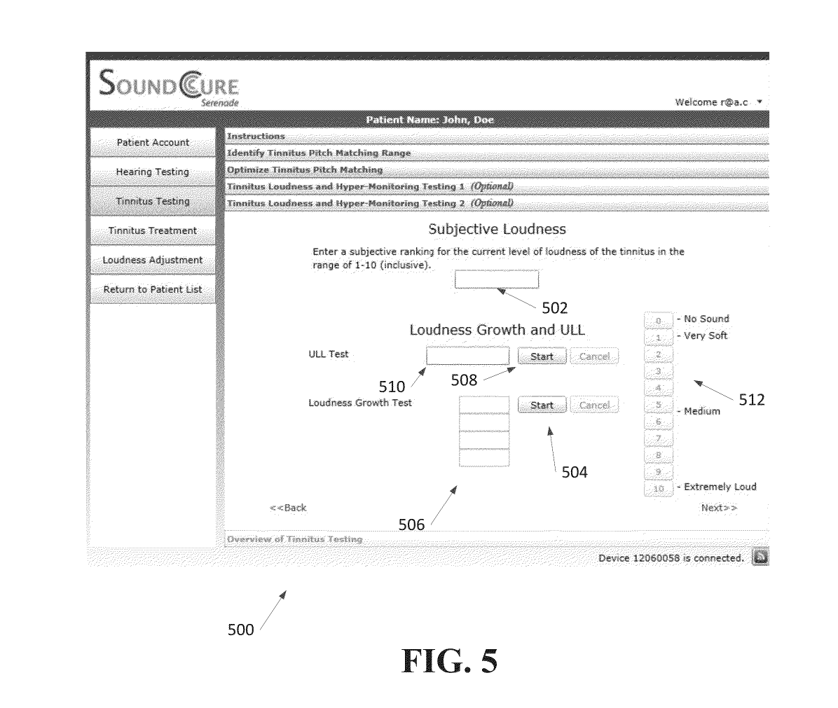

A system 100 of FIG. 1 uses an audiometer for evaluating hypermonitoring of tinnitus of a patient. The system 100 includes a processor 102 for controlling an audiometer 104 and for accessing a memory device 106 storing executable instructions. The processor 102 executes the executable instructions stored on the memory device 106 to control the audiometer and in response to operator input to evaluate tinnitus of a patient 112. The system can include a display 108 driven by the processor 102 providing screen shots (e.g., see FIGS. 4-5) to an operator 114 of the system. The operator 114 uses an input device 110 to provide input information to the processor 102 to control the processor and tinnitus testing. The input device 110 may be any one or more of a keypad, keyboard, mouse, track ball, audio translator and/or any other input which allows the operator 114 to provide instructions and parameters to the processor 102 to control the tinnitus testing.

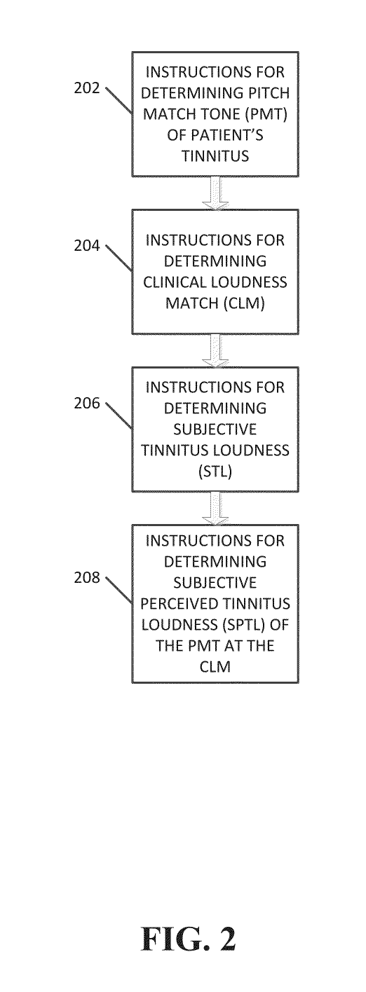

Referring to FIG. 2, computer executable instructions stored on the memory device 106 for execution by the processor 202 include instructions 202 for determining a pitch match tone (PMT) of the tinnitus of the patient 112; instructions 204 for determining a clinical loudness match (CLM) of the tinnitus of the patient 112; instructions 206 for determining a subjective tinnitus loudness (STL) of the patient's tinnitus; and instructions 208 for determining a subjective perceived tinnitus loudness (SPTL) of the patient 112 of the PMT 202 at the CLM 204.

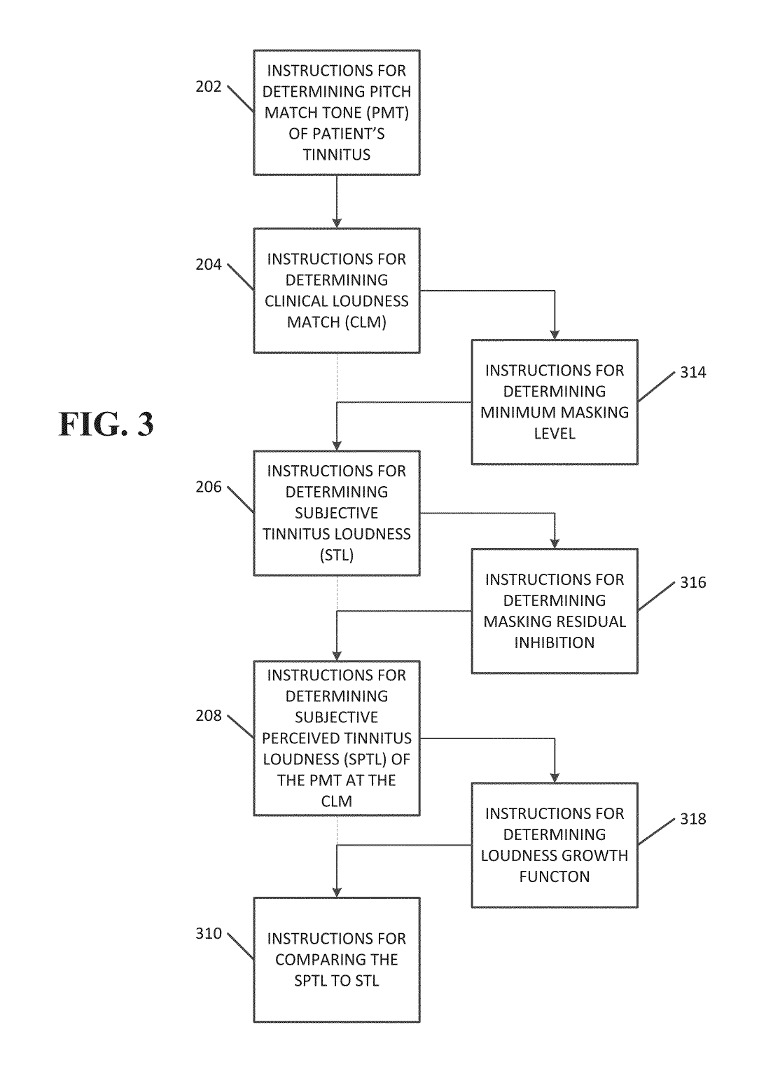

The executable instructions may include one or more additional aspects as illustrated in FIG. 3, such as instructions 310 for comparing the SPTL 208 to the STL 206; instructions 314 for determining a minimum masking level of the patient 112; instructions 316 for determining a masking residual inhibition of the patient 112; and/or instructions 318 for determining a loudness growth function of the patient 112.

The goal of the pitch match tone (PMT; also referred to as a pitch match threshold) instructions 202 is to determine the closest pure tone match to the patient's tinnitus. A pitch match tone/threshold can be used to show a correlation with damage to the auditory system. Damage can be shown from behavioral audiometry or, in the case of normal hearing, otoacoustic emissions (OAEs) or extended high frequency thresholds may be used.

Preferably, the instructions 202 begin by directing the operator 114 to perform a pitch match after pure tone thresholds are known. Thus, a pure tone threshold and a pitch match are determined by the following instructions and screen shots which are executed by the processor to control the audiometer and to direct the operator 114 through the following process. A tone (e.g., 1 kHz at 10 dB SL) is presented to the patient 112 and the operator 114 is prompted to ask the patient if the tone is higher or lower in pitch than the patient's tinnitus, ignoring loudness differences. Random tones at octave, half octave or other intervals are presented to the patient and the operator 114 is prompted to categorize the patient's response. The processor 102 controls the audiometer 102 to change tones in either a consistent manner according to the patient's response (i.e. making it higher if they state it is too low, or lower if they state it is too high) or in a random or pseudo-random manner so as not to steer the patient's decision. If the patient reports that he/she is unsure about the pitch of his/her tinnitus relative to the tone, an alternative choice paradigm may be implemented. For example, the processor 102 may present two different tones sequentially and ask which is more similar. If a number of tones are judged equally similar, the processor 102 prompts the operator 114 to use the input device 110 to indicate an average, a central choice from the range, or any value within the matched range for the match.

For example, as illustrated in FIG. 4, the operator 114 is presented on display 108 with a screen shot 400 which assists the operator 114 in determining the pitch match threshold of the patient 112. The operator 114 selects start button 402 which causes the processor 102 to control the audiometer 104 to present tones to the patient 112. The operator 114 enters the patient's response using button 404 if the patient hears a tone and button 406 if the patient hears no sound. When the test is complete, the pitch match threshold is calculated by the processor and indicated in box 408.

Thus, the instructions 202, 204, 206, 208 as illustrated in FIG. 2 can include instructions to present varying tones to the patient; instructions for increasing a pitch of the tones when the patient indicates that the tones are lower in pitch than the patient's tinnitus; and instructions for decreasing a pitch of the tones when the patient indicates that the tones are greater in pitch than the patient's tinnitus.

There are other processes that the processor 102 may implement in order to collect a threshold of a tone. For example, instructions implementing the Hughson-Westlake procedure may be stored in memory 106 for execution by processor 102.

A clinical loudness match (CLM) can be compared to the patient's threshold at the pitch match tone (PMT). Typically, matches are less than 20 dB SL and are very commonly only a few dB. When possible, a loudness match is helpful in demonstrating to a patient that the patient's tinnitus is soft. Often with sensorineural hearing loss, a low number in dB above threshold does not mean soft tinnitus. One way to determine this is a loudness growth function 318 (see below).

The instructions 204 executed by the processor 102 for controlling the audiometer 104 to obtain a CLM can include the following. Using the pitch match tone (PMT), the processor 102 instructs the audiometer 104 to present a tone at the pitch match threshold to the patient 112, and the operator 114 uses the input device 110 to input the patient's response, reporting if the presented tone is louder or softer than the patient's tinnitus. The processor 102 varies the loudness level (e.g., with a 5 dB step size first and then a 1 dB step size). Optionally, the processor can implement starting low and increasing in 1 dB steps to avoid residual inhibition. Instructions 204 may be executed by the operator 114 selecting a start button 410 in FIG. 4. The operator 114 uses buttons 412 to enter the patient's response of louder than, similar to, or softer than the patient's tinnitus. The CLM is found as the average of a number of reversal points (i.e. points in which the patient's response switches from louder to softer, or softer to louder) and is indicated in box 414 when the test is complete and its corresponding standard deviation (abbreviated SD) is calculated by the processor 102 and indicated in box 416.

The memory device 106 includes instructions 206 which prompt the operator 114 to assist the patient in determining a subjective tinnitus loudness (STL) of the patient's tinnitus. In this determination, the operator 114 is prompted to present to the patient 112 a subjective scale (e.g., 0-10 or whisper, soft, medium, loud, very loud) and the operator 114 asks the patient 112 to provide an STL value corresponding to the loudness of their tinnitus based on the subjective scale. The operator 114 is prompted to enter the value into the system 100 using the input device 110. For example, as illustrated in FIG. 5, the operator 114 is presented on display 108 with a screen shot 500 which assists the operator 114 in determining the patient's 112 subjective loudness (STL) of their tinnitus. In box 502, the operator 114 enters the patient's STL value.

The memory device 106 includes instructions 208 for determining a subjective perceived tinnitus loudness (SPTL) of the patient 112 of the PMT 202 at the CLM 204. The processor 102 controls the audiometer 104 to present to the patient a tone of the PMT 202 at the CLM 204. In this determination, the operator 114 is prompted to again present to the patient 112 the same subjective scale (e.g., 1-10 or whisper, soft, medium, loud, very loud) and the operator 114 asks the patient 112 to provide an SPTL value corresponding to the tone of the PMT 202 at the CLM 204 based on the subjective scale. The operator 114 is prompted to enter the SPTL value into the system 100 using the input device 110.

Thus, the instructions 202 can comprise Instructions to present the pitch match tone (PMT) at varying loudness levels to the patient; instructions for increasing a volume of the PMT when the patient indicates that the PMT is lower in loudness than the patient's tinnitus; and instructions for decreasing a volume of the PMT when the patient indicates that the PMT is greater in loudness than the patient's tinnitus.

Optionally, the processor 102 presents a screen to the operator 114 allowing a comparison of the SPTL value to the STL value. A discrepancy between the perceived loudness (STL value) and more objective loudness (SPTL value) is an indication of hypermonitoring. The value of this test is both in counseling a patient about how sound therapy works, in that it corrects this perceptual mismatch when present, and can be used to counsel patients about sound therapy. For example, patients who are hypermonitoring can achieve improvement through the process of habituation as this perceptual mismatch is corrected.

The minimum masking level (MML) 314 is the loudness at which noise is presented such that the patient's tinnitus cannot be heard. Typically MML is performed with noise such as, for example, measuring with narrowband noise centered at the tinnitus pitch match tone PMT. The MML is an optional tool for determining if white noise or narrowband noise is an option for sound therapy. A low level indicates it is likely an option. The optional instructions 314 may be implemented between instructions 204 and 206, or at some other point, and include the processor 102 controlling the audiometer by presenting noise at or below MML and having the operator 114 ask the patient if it is tolerable.

In general, different levels of precision may be used. Frequently, 1 dB precision or 5 dB may provide the appropriate results, although other levels of precision may be used. Many patients have MMLs of 50-70 dB, so the processor may be set to provide a variable (e.g., 5 dB) step size, depending on the patient, the operator and the time and efficiency of the testing. Along with MML, finding a point in which the tinnitus is ‘mostly’ obscured, or a point of sufficient relief, has value since many patients find a low level masker blocks the majority of their tinnitus, but that complete masking requires much louder stimuli.

Optionally, the processor 102 measures residual inhibition (RI) by controlling the audiometer to present noise (e.g., white noise) to the patient 112 (unless patient is intolerant). The optional instructions 316 may be implemented between instructions 206 and 208, or at some other point, and include the processor 102 controlling the audiometer by presenting noise to the patient 112 (e.g., white noise at MML+10 dB) for a preset period (e.g., 60 seconds) unless the patient is intolerant. At the end of the preset period, the operator 114 is instructed to ask the patient 112 if he/she can hear his/her tinnitus, and if so, if it is reduced. The time required for the tinnitus to return to normal is recorded by the operator 114 using the input device 110. Since the phenomenon of RI has little bearing on long term habituation, it is optional.

Measuring an optional loudness growth function at the tinnitus pitch match can give a semi-objective measure of the loudness of the patient's tinnitus. Comparing this rating to the subjective tinnitus loudness rating can give a measure of hypermonitoring. The loudness growth function may assist in interpreting the loudness match. The instructions 318 include asking the patient how loud the tinnitus is on a 0-10 scale making sure the patient is not giving a measure of ‘severity’ or ‘annoyance’; presenting tones at four levels: the pitch match at threshold, the clinical loudness match, and two levels above the clinical loudness match (e.g., 5 or 10 dB above the CLM); and asking the patient to rank the loudness on the same 0-10 scale for each presentation.

Sounds may be presented multiple times and averaged, either by the operator 114 or by the processor 104. The difference between the loudness ranking of the loudness match for the tinnitus pitch and the subjective ranking can be presented to the operator 114 on display 108. The difference is indicative of the magnitude of hypermonitoring. The magnitude of hypermonitoring can be an indicator of the perceptual part of tinnitus. For example, if a patient is not hypermonitoring, they may potentially be a tinnitus management patient and possibly less of a habituation patient—although all patients can potentially benefit from long-term sound therapy management. The loudness growth function instructions 318 may be implemented between 208 and 310, or at some other point.

For example, as illustrated in FIG. 5, the operator 114 is presented on display 108 with a screen shot 500 which assists the operator 114 in determining the patient's loudness growth and upper loudness levels (ULL). By selecting the start button 504, the processor 102 executes the instructions 218. In boxes 506 are the four levels of tones presented. These four levels can be entered by the operator 114 or indicated by the processor 102. For each tone level, the operator 114 selects one of the buttons 512 labeled 0-10 as indicated by the patient 112. In box 502, the operator 114 enters the patient's STL value. Optionally, the operator 114 can determine the patient's upper loudness levels (ULL) by selecting the start button 508. The processor 112 executes instructions to vary tones presented to the patient 112 to determine the patient's ULL, which appears in box 510. Once again, the operator 114 uses buttons 512 to indicate the patient's response to the varying tones.

### EXAMPLE

A patient has tinnitus that matches to 8000 Hz. The patient's threshold at 8000 Hz is 35 dB HL and his tinnitus matches to 45 dB HL. As outlined above, it is unclear what 45 dB HL sounds like to this patient. When a 45 dB HL tone at 8000 Hz is presented to the patient, he reports that it is soft at a level of 3 on a 10 point scale. He rates his tinnitus loudness at 7 out of 10. This discrepancy indicates that he is hypermonitoring.

In summary, the following describes the operation of the processor executing the instructions to implement the testing process and presenting the accompanying screen shots; the order of the process is variable. Subjective perceived loudness and subjective loudness can be obtained and compared.

A pitch match is performed to the patient's tinnitus as is conducted clinically.

The patient's threshold of his pitch match is found. The loudness match of the patient's tinnitus is found as per typical clinical assessment. There is no recognized standard for finding a match. There are a number of ways to find this. For example, an algorithm in the software may be used.

The subjective loudness is determined. This is simply the patient's report of the loudness of the patient's tinnitus. For example, a 10 point scale may be used, but other scales could be used. One such scale is a visual analog scale in which a line is drawn by the patient on an unmarked axis and the length from the end is measured. The patient can be instructed to report on loudness and not on severity, annoyance, distress or some other parameter.

The subjective perceived loudness of the loudness match is determined in several ways. For example, one method is to present the pitch match tone at the loudness match to the patient and ask how loud this tone is on the same scale used above. Practically, presenting more than one tone is preferable. For example, four tones—one at threshold, one at the loudness match and two above the match—may be used. Any number of tones may be used along with using tones at different pitches along with the pitch match.

The subjective tinnitus loudness (STL) is compared to the subjective perceived tinnitus loudness (SPTL) of the loudness match. If they are different, the patient is hypermonitoring. If they are the same, the patient perceives his tinnitus correctly.

If a patient is hypermonitoring, an understanding of this helps in the treatment of tinnitus by allowing the patient to know how sound therapy works by correcting the patient's perception of her tinnitus. Further, hypermonitoring is believed to be a precursor to habituation. Habituation is the process by which a patient gradually perceives their tinnitus less over time. Therefore, patients who are hypermonitoring can begin sound therapy treatment with this expectation.

If a patient is not hypermonitoring, the patient's tinnitus may be soft and she perceives it soft. In case, she may not be a candidate for treatment of her tinnitus, at the time of the test, because her tinnitus is not bothersome. Alternatively, her tinnitus may be loud and she perceives it loud and she may not be a candidate for habituation but is a candidate for tinnitus management. In some cases, the patient is more likely to benefit from sound therapy, much in the way hearing aids are used chronically.

Optionally, the system and method can be used with implants, so that as used herein generating, applying or subject a patient to a sound or tone includes applying an electrical stimulation to the patient to simulate or otherwise indicate a sound or tone.

The Abstract and summary are provided to help the reader quickly ascertain the nature of the technical disclosure. They are submitted with the understanding that they will not be used to interpret or limit the scope or meaning of the claims. The summary is provided to introduce a selection of concepts in simplified form that are further described in the Detailed Description. The summary is not intended to identify key features or essential features of the claimed subject matter, nor is it intended to be used as an aid in determining the claimed subject matter.

For purposes of illustration, programs and other executable program components, such as the operating system, are illustrated herein as discrete blocks. It is recognized, however, that such programs and components reside at various times in different storage components of a computing device, and are executed by a data processor(s) of the device.

Although described in connection with an exemplary computing system environment, embodiments of the aspects of the invention are operational with numerous other general purpose or special purpose computing system environments or configurations. The computing system environment is not intended to suggest any limitation as to the scope of use or functionality of any aspect of the invention. Moreover, the computing system environment should not be interpreted as having any dependency or requirement relating to any one or combination of components illustrated in the exemplary operating environment. Examples of well-known computing systems, environments, and/or configurations that may be suitable for use with aspects of the invention include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

Embodiments of the aspects of the invention may be described in the general context of data and/or processor-executable instructions, such as program modules, stored one or more tangible, non-transitory storage media and executed by one or more processors or other devices. Generally, program modules include, but are not limited to, routines, programs, objects, components, and data structures that perform particular tasks or implement particular abstract data types. Aspects of the invention may also be practiced in distributed computing environments where tasks are performed by remote processing devices that are linked through a communications network. In a distributed computing environment, program modules may be located in both local and remote storage media including memory storage devices.

In operation, processors, computers and/or servers may execute the processor-executable instructions (e.g., software, firmware, and/or hardware) such as those illustrated herein to implement aspects of the invention.

Aspects of the invention may be implemented with processor-executable instructions. The processor-executable instructions may be organized into one or more processor-executable components or modules on a tangible processor readable storage medium. Aspects of the invention may be implemented with any number and organization of such components or modules. For example, aspects of the invention are not limited to the specific processor-executable instructions or the specific components or modules illustrated in the figures and described herein. Other embodiments of the aspects of the invention may include different processor-executable instructions or components having more or less functionality than illustrated and described herein.

The order of execution or performance of the operations of the invention illustrated and described herein is not essential, unless otherwise specified. That is, the operations may be performed in any order, unless otherwise specified, and aspects of the invention may include additional or fewer operations than those disclosed herein. For example, it is contemplated that executing or performing a particular operation before, contemporaneously with, or after another operation is within the scope of the invention.

When introducing elements of the invention, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that several advantages of the aspects of the invention are achieved and other advantageous results attained.

Not all of the depicted components illustrated or described may be required. In addition, some implementations may include additional components. Variations in the arrangement and type of the components may be made without departing from the spirit or scope of the claims as set forth herein. Additional, different or fewer components may be provided and components may be combined. Alternatively or in addition, a component may be implemented by several components.

The above description illustrates the aspects of the invention by way of example and not by way of limitation. This description enables one skilled in the art to make and use the aspects of the invention, and describes several adaptations, variations, alternatives and uses of the aspects of the invention, including what is presently believed to be the best mode of carrying out the aspects of the invention. Additionally, it is to be understood that the aspects of the invention are not limited in their application to the details of construction and the arrangement of components set forth in the detailed description or illustrated in the drawings. The aspects of the invention are capable of other embodiments and of being practiced or carried out in various ways. Also, it will be understood that the phraseology and terminology used herein is for the purpose of description and should not be regarded as limiting. It will, however, be evident that various modifications and changes may be made thereto, and additional embodiments may be implemented, without departing from the broader scope of the aspects of the invention as set forth in the claims that follow. The specification and drawings are accordingly to be regarded in an illustrative rather than restrictive sense.

What is claimed is:

1. A system 100 for use with an audiometer 104 for evaluating hypermonitoring of tinnitus of a patient 112, said system 100 comprising:
a memory device 106 for storing executable instructions;
a processor 102 adapted for controlling the audiometer 104, adapted for accessing the memory device 106 and adapted to execute the executable instructions stored on the memory device 106;
a display 108 driven by the processor 102 for providing screen shots to an operator 114 of the system 100; and
an input device 110 for use by the operator 114 for providing input information to the processor 102;
wherein said executable instructions stored in the memory device 106 comprise:
instructions 202 for determining a pitch match tone (PMT) of the patient's tinnitus;
instructions 204 for determining a clinical loudness match (CLM) of the patient's tinnitus;
instructions 206 for determining a subjective tinnitus loudness (STL) of the patient's tinnitus; and
instructions 208 for determining a subjective perceived tinnitus loudness (SPTL) of the patient's PMT at the CLM.

2. The system of claim 1 wherein said executable instructions further comprise instructions 310 for comparing the SPTL to the STL.

3. The system of claim 1 wherein the instructions 202 for determining a PMT of the patient's tinnitus comprise:
instructions to present varying tones to the patient;
instructions for increasing a pitch of the tones when the patient indicates that the tones are lower in pitch than the patient's tinnitus; and
instructions for decreasing a pitch of the tones when the patient indicates that the tones are higher in pitch than the patient's tinnitus.

4. The system of claim 3 wherein instructions 204 for determining a CLM of the patient's tinnitus comprise:
instructions to present the pitch match tone PMT at varying loudness levels to the patient;
instructions for increasing a volume of the PMT when the patient indicates that the PMT is lower in loudness than the patient's tinnitus; and
instructions for decreasing a volume of the PMT when the patient indicates that the PMT is higher in loudness than the patient's tinnitus.

5. The system of claim 4 wherein instructions 208 for determining the SPTL of the patient's PMT at the CLM comprise instructions to present to the patient a tone of the PMT at the CLM.

6. The system of claim 5 further comprising instructions for comparing the SPTL value with the STL value indicated by the patient.

7. The system of claim 1 further comprising instructions 314 for determining a minimum masking level (MML) of the patient 112 comprising presenting noise at various loudness levels to the patient to determine which presented noise loudness level masks the patient's tinnitus so that the patient's tinnitus cannot be heard by the patient.

8. The system of claim 7 further comprising instructions 316 for determining a residual inhibition (RI) of the patient 112 comprising instructions for presenting to patient 112 noise for a preset period of time to determine whether the patient hears the tinnitus thereafter.

9. The system of claim 4 further comprising instructions 318 for determining a loudness growth function of the patient 112 comprising instructions for presenting to the patient tones at the PMT, the CLM and at two other loudness levels for comparison to a difference value indicative of hypermonitoring.

10. The system of claim 1 further comprising instructions for determining an upper loudness level (ULL) of the patient 112 wherein the processor executes the ULL instructions to control the audiometer to present various tones to the patient.

11. A processor executable method for use with an audiometer for evaluating hypermonitoring of tinnitus of a patient, said method comprising processor executable instructions for controlling the audiometer, said processor executable method comprising:
determining 202 a pitch match tone (PMT) of the patient's tinnitus;
determining 204 a clinical loudness match (CLM) of the patient's tinnitus;
determining 206 a subjective loudness (STL) of the patient's tinnitus; and
determining 208 a subjective perceived loudness (SPTL) of the patient of the PMT at the CLM.

12. The processor executable method of claim 11 wherein said executable instructions further comprise comparing 310 the SPTL to the STL.

13. The processor executable method of claim 11 wherein determining 202 a PMT of the patient's tinnitus comprises:
presenting varying tones to the patient;
increasing a pitch of the tones when the patient indicates that the tones are lower in pitch than the patient's tinnitus; and
decreasing a pitch of the tones when the patient indicates that the tones are higher in pitch than the patient's tinnitus.

14. The processor executable method of claim 13 wherein determining 204 a CLM of the patient's tinnitus comprises:
presenting the pitch match tone PMT at varying loudness levels to the patient;
increasing a volume of the PMT when the patient indicates that the PMT is lower in loudness than the patient's tinnitus; and
decreasing a volume of the PMT when the patient indicates that the PMT is higher in loudness than the patient's tinnitus.

15. The processor executable method of claim 14 wherein determining 208 the SPL of the patient's PMT at the CLM comprises presenting to the patient a tone of the PMT at the CLM.

16. The method of claim 11 further comprising determining 314 a minimum masking level (MML) of the patient 112 comprising presenting noise at various loudness levels to the patient to determine which presented noise loudness level masks the patient's tinnitus so that the patient's tinnitus cannot be heard by the patient.

17. A tangible computer readable storage media storing processor executable instructions for use with an audiometer for evaluating hypermonitoring of tinnitus of a patient, said processor executable instructions adapted for controlling the audiometer, said media comprising:
instructions 202 for determining a pitch match tone (PMT) of the patient's tinnitus;
instructions 204 for determining a clinical loudness match (CLM) of the patient's tinnitus;
instructions 206 for determining a subjective loudness (STL) of the patient's tinnitus; and
instructions 208 for determining a subjective perceived loudness (SPTL) 208 of the patient of the PMT at the CLM.

18. The media of claim 17 wherein the instructions 202 for determining a PMT of the patient's tinnitus comprise:
instructions to present varying tones to the patient;
instructions for increasing a pitch of the tones when the patient indicates that the tones are lower in pitch than the patient's tinnitus; and
instructions for decreasing a pitch of the tones when the patient indicates that the tones are higher in pitch than the patient's tinnitus.

19. The media of claim 17 wherein the instructions 204 for determining a CLM of the patient's tinnitus comprise:
instructions to present the pitch match tone PMT at varying loudness levels to the patient;
instructions for increasing a volume of the PMT when the patient indicates that the PMT is lower in loudness than the patient's tinnitus; and
instructions for decreasing a volume of the PMT when the patient indicates that the PMT is higher in loudness than the patient's tinnitus.

20. The media of claim 17 further comprising instructions 310 for comparing the SPL to the SL and wherein the instructions 208 for determining the SPL of the patient's PMT at the CLM comprise instructions to present to the patient a tone of the PMT at the CLM.
